(19) United States
(12) Patent Application Publication
(10) Pub. No.: US 2005/0147720 A1
(43) Pub. Date: Jul. 7, 2005

(54) Method of analyzing protein

(76) Inventors: Naoyuki Yamada, Kanagawa (JP); Shinichi Ozawa, Kanagawa (JP); Naoko Kageyama, Kanagawa (JP); Hiroshi Miyano, Kanagawa (JP)

Correspondence Address: CERMAK & KENEALY LLP;ACS LLC, 515 EAST BRADDOCK ROAD, SUITE B, ALEXANDRIA, VA, 22314, US

(21) Appl. No.: 11/039,791

(22) Filed: Jan. 24, 2005

Related U.S. Application Data
(63) Continuation of application No. PCT/JP03/09305, filed on Jul. 23, 2003.

(30) Foreign Application Priority Data
Jul. 25, 2002 (JP) 2002-217099

(51) Int. Cl. G01N 33/02

(52) U.S. Cl. 426/231

(57) ABSTRACT

A sample is analyzed for trace amounts of protein by dot blotting and subsequent fluorescence staining. According to this method, trace amounts of proteins such as an allergen in a food, a drink, a food additive, a medicament and feed etc. can be simply analyzed with high sensitivity. By providing the objective analysis method as described above, quantitative analysis and limit analysis of a protein contained in a sample can be performed.

This application is a continuation of application PCT/JP03/09305, filed on Jul. 23, 2003.

### BACKGROUND OF THE INVENTION

1. Field of the Invention

The present invention relates to a novel method of analyzing a protein contained in food, drinks, food additives, medicaments, feed, etc..

2. Brief Description of the Related Art

Impurities contained in food and food additives are often allergenic substances, which has recently become more problematic. To prevent health injuries caused by allergen-containing foods, the Sanitary Act of Japan was revised in April 2001. This ordinance imposes an obligation to notify of the allergenic substance in five specific raw materials—egg, milk, wheat, buckwheat, and peanut. Since the allergenic substance is a protein, the Ministry of Labor, Health and Welfare has published a guideline stating that foods having a total protein content of at least a few μg/g require notification of any allergic substances therein (see “Interim Report of the Allergy Expression Investigation Society of the Food Expression Study Group” by the Planning Section of the Food Insurance Department of the Drug Bureau of the Ministry of Labor, Health and Welfare, dated Oct. 29, 2001).

Enzyme linked immunosorbent assay (ELISA) is generally employed for the detection of trace amounts of allergenic substances in foods or food additives. In March 2002, just before the enforcement of the ministerial ordinance, Nippon Ham Co. sold ELISA kits for detection of the 5 items listed above. Similarly, on Mar. 27, 2002, Snow Brand Milk Products Co. announced in the Agricultural Chemistry Society of Japan (Annual Meeting of the Agricultural Chemistry Society of Japan, 2002) a method of performing ELISA for detection of soybean protein. The method comprises extracting a protein from a raw material using a physiological saline solution, immunizing rabbits with the extract, and establishing an ELISA system using IgG roughly purified through a protein A column. The sensitivity of the method is high, at the level of 1 ng/ml for soybean, but since the degree of purification of the material and the antibody is low, the selectivity of soybean protein is low in this method. In addition, there is no information relating to the antigen. In the ELISA method, the antibody to the allergen (antigen) must be prepared first, and then the analytical method must be established, which can be time-consuming. In addition, there is no guarantee that all allergens will be detected. Furthermore, another problem exists in that the sensitivity and the specificity fluctuate depending on the properties of the antibody used. Moreover, since an antigen-antibody reaction is used in the ELISA method, a neutral solution that enables antigen-antibody bonding must be employed as the sample solution. However, some solid samples do not always have a high solubility at around a neutral pH. For example, some amino acids are hard to dissolve at around a neutral pH. Tyrosine has an extremely low solubility at neutral pH, and therefore the protein detection sensitivity per weight could be from tens to hundreds of ppm, even in ELISA. Moreover, the methods for detection of allergen have many problems. (see R. E. Poms “Methods for allergan analysis in food: a review,” Food Additivies and Contaminants, 21(1):1-31 (2004))

Alternatively, a method has been reported combining a dot blot and staining. For example, in dot blotting, in general, the protein that is concentrated and fixed on a membrane can be visualized by staining with Coomassie brilliant blue, or the like. Another known staining method having a high-sensitivity is gold-colloid staining, which, has many drawbacks, however, such as a high background, staining unevenness, and the operation is complicated.

Therefore, a method capable of analyzing trace amounts of a protein in a simple manner with high reproducibility and with high sensitivity is desired and needed.

### SUMMARY OF THE INVENTION

The present invention provides an analytical method for measuring the amount of a protein or proteins, such as allergic substances, contained in trace amounts in foods, drinks, food additives, medicaments, feeds, etc. This novel method is simple, has a high reproducibility, and has high sensitivity. The present inventors have found that combining a dot blotting method and a fluorescent staining method heretofore employed in the field of biochemistry results in the ability to measure trace amounts of proteins in various samples.

It is an object of the present invention to provoide a method of analyzing a sample for allergenic substances comprising
a) dissolving said sample in an aqueous solution,
b) fixing protein in said solution onto a membrane, and
c) detecting the level of said protein by fluorescence staining, wherein the level of said protein indicates the presence of said allergenic substance.

It is a further object of the present invention to provide the method as described above, wherein the presence of said allergenic substance is determined by detection of trace amounts of said protein.

It is still a further object of the present invention to provide the method as described above, wherein 0.1 ppm of said protein is able to be detected.

It is a further object of the present invention to provide the method as described above, wherein said membrane is selected from the group consisting of nitrocellulose and PVDF.

It is a further object of the present invention to provide the method as described above, wherein said sample is selected from the group consisting of food, drinks, food additives, medicaments, feed.

It is a further object of the present invention to provide the method as described above, wherein said sample is an amino acid.

It is a further object of the present invention to provide the method as described above, wherein the solubility of said amino acid is improved by dissolving said amino acid in an aqueous solution with an acidic pH.

### BRIEF DESCRIPTION OF THE DRAWINGS

FIG. 1 shows an outline of the dot blotting apparatus used in Example 1. is the sample template; (2) is the gasket; (3) is the gasket support plate; (4) is the vacuum manifold; (5) is the membrane; and (6) is the filter paper.

FIG. 2 shows image data of BSA (bovine serum albumin) preparation and amino acid+BSA sample in Example 1.Lanes 1 and 2 are amino acid+BSA sample; Lanes 3 and 4 are the BSA preparation.

FIG. 3 shows a calculation method of concentration of a protein in amino acid according to a standard addition method in Example 1. ●: Amino acid; ▪: BSA; Arrow: Protein concentration in amino acid solution; Horizontal axis: Amount of BSA added; Vertical axis: Fluorescence intensity.

FIG. 4 shows the result of measurement of protein (standard addition method) in “Glu-Na” (sodium L-glutamate monohydrate) and “The” (L-theanine), according to a dot blotting-fluorescent staining method in Example 1. ●: BSA; ▪: Glu-Na; ▴: The. Horizontal axis: BSA concentration (ppm); Vertical axis: Fluorescence intensity.

FIG. 5 shows the result of measurement of protein in BSA, lysozyme, ubiquitin, insulin and oxidized insulin B-chain, according to a dot blotting-fluorescent staining in Example 2. ●: BSA; ▪: Insulin; ▴: ubiquitin; ×insulin-oxidized B; +: lysozyme. Horizontal axis: Protein concentration (ppm); Vertical axis: Fluorescence intensity.

### DESCRIPTION OF THE PREFERRED EMBODIMENTS

The present invention provides an analytical method for determining trace amounts of proteins, such as allergenic substances, contained in food, drinks, food additives, medicaments, feed, etc., which is simple, highly reproducible, and very sensitive. The present inventors have found that when a dot blotting method and a fluorescent staining method, typically employed in the field of biochemistry, are combined, trace amounts of proteins in a sample can be simply analyzed with high sensitivity.

Therefore, one embodiment of the present invention is a method of analyzing a sample for protein content by subjecting a sample to dot blotting, and subsequent fluorescence staining. The analytical method of the present invention enables quantitative analysis and limit analysis (limit test) of the protein contained in a sample.

The method may include a step of subjecting the sample in a solution to the dot blotting to thereby fix the protein on a membrane which is used for protein fixation in the sample, followed by staining the protein fixed on the membrane by fluorescence staining.

The sample used in the present invention may be any food, drink, food additive, medicament, feed, and/or intermediates of these. One preferred example is an amino acid supplement, typically used as a food additive. The method has a sufficient sensitivity for detection of proteins contained in trace amounts in such products.

According to the method of the present invention, at least 0.1 ppm of protein can be preferably analyzed.

The membrane used in the present invention for protein fixation is preferably a nitrocellulose membrane and/or a polyvinylidene difluoride, hereinafter “PVDF”, membrane.

Another embodiment of the present invention includes products in which the protein has been analyzed according to the method of the present invention, for example, foods, drinks, food additives, medicaments, feeds and their intermediate products, as well as amino acids, etc. Naturally, further, the present invention encompasses the products thus analyzed in the manner as above, or those using them (products).

The mode for carrying out the invention is described below.

In the present invention, the sample to be analyzed is applied to a membrane to thereby fix any protein in the sample onto the membrane, according to a dot blot method. In this case, in general, a solution of the sample is subjected to fixation.

One typical example of the dot blot method involves putting, for example, a nitrocellulose membrane, a PVDF membrane, or the like, between acrylic plates having a large number of holes, then injecting a protein solution into a hole on the membrane, and subjecting it to suction filtration by aspiration, resulting in fixation of the protein onto the membrane. According to the method, it is possible to fix and hold a large number of different types of protein samples on a membrane while concentrating and desalting them (see Hirano, Protein Structure Analysis for Gene Cloning, Blotting and Sequencing, (Tokyo Kagaku Dojin), p. 62).

The next step is staining the membrane containing the fixed protein by fluorescence staining.

A fluorescent staining reagent is SyproRuby, recently developed for proteome analysis for protein gel separated through SDS electrophoresis. The advantage of this reagent is that it enables rapid and simple staining with high sensitivity for accurate quantification as compared with conventional methods. This reagent may be used, for example, for detection or quantification of a protein fixed on a PVDF membrane. In protein quantification at a higher sensitivity, the loss may be greater due to adsorption to vessels or the like in typical operations, and therefore, detection may be difficult. Accordingly, the present inventors have further investigated detecting trace amounts of protein, and, as a result, have established a high-sensitivity method of quantifying and analyzing protein in which the dilution operation is reduced as much as possible and siliconized containers and chips are used which enables detection of 0.1 ppm-level protein.

According to the method of the present invention, the combination of dot blotting and fluorescent staining provides a method of analyzing trace amounts of protein in a sample within a relatively short period of time (approximately 4 or 5 hours from the sample preparation and the image analysis), and with high sensitivity, providing for accurate quantification. Although the method of the present invention may be somewhat inferior as compared to ELISA for sensitivity, it is far superior to ELISA in that all the proteins fixed on the membrane can be detected and determined in the method of the present invention. Furthermore, smaller, highly hydrophobic proteins can be detected, particularly when preferred membranes for protein fixation are selected or developed. Detection of solid samples is possible when the sensitivity per weight is higher because then their solubility is higher. In the present invention, since the pH of the solvent is not specifically defined, any acid, neutral or alkaline solvent may be used. Therefore, samples may be dissolved in a solvent having a pH value that is the most suitable for the solubility of the samples, and, as a result, the detection sensitivity can be improved.

Quantification and specificity in ELISA method significantly depends on the properties of the antibody used, and it is extremely difficult to completely determine all proteins or peptides that may be antigens with no detection failure. In addition, antigens cannot be detected when the antibodies to them are not prepared. Conversely, the dot blotting-fluorescent staining method according to the present invention enables detection of all proteins, and therefore enables evaluation of the proteins irrespective of the presence or absence of antigenicity in protein. Accordingly, the method according to the present invention is a measurement method of higher reliability.

The process of the present invention is described briefly. A solution sample can be used as is. However, a solid sample should be dissolved in a solution having a high solubility to give the highest possible concentration. Amino acid samples have a high solubility in acid solutions. Therefore, it is desirable that amino acid samples are dissolved in aqueous 3.5% hydrochloric acid solution. For accurately determining the amount of protein in samples according to a standard addition method, samples with a standard protein (e.g., bovine serum albumin, BSA) suitably added thereto are prepared. Next, the protein in the sample is trapped, or fixed, on a nitrocellulose membrane or a PVDF membrane and, measurement-disturbing substances are removed at the same time from the sample. A 96-well dot blotting apparatus can be purchased commercially so that multiple samples can be processed at a time.

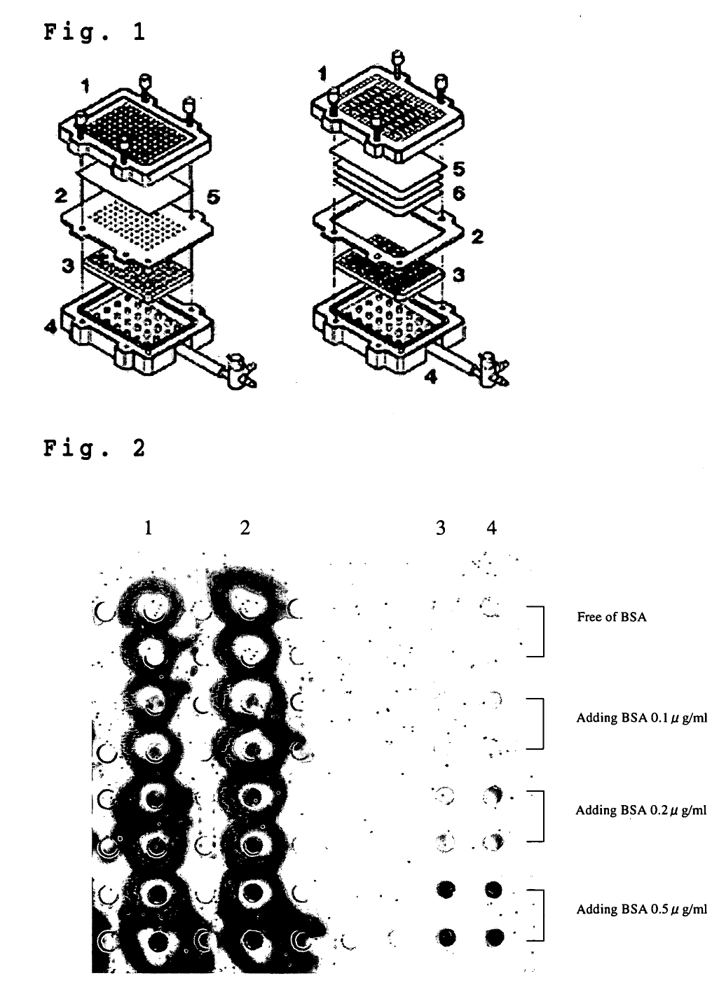

After the sample is injected, an aspirator is used to absorb the protein in the sample onto the membrane for protein fixation. A PVDF membrane of high absorbability is preferable. After the liquid has been completely aspirated away, water is injected a few times, and then aspirated, whereby the analysis-disturbing substances remaining on the membrane can be removed. Regarding amino acid samples, amino acids reacting with the staining solution, as well as disturbing low-molecular substances and hydrochloric acid and the like can be removed. Next, the washed membrane is taken out of the blotting apparatus (dot blotting apparatus, see FIG. 1), and sufficiently dried.

The membrane dried as described above is then stained with a fluorescent staining reagent. The fluorescent staining reagent SyproRuby (manufactured by Molecular Dynamics) was developed for proteome analysis and is a reagent of high sensitivity and good quantification. This reagent is prefered.

The dry membrane is then subjected to treatment with an acetic acid-methanol solution, whereby the protein is fixed on this membrane, and then stained with a fluorescent staining reagent. Next, the membrane is washed, and then analyzed preferably using a fluorescent image analyzer.

With the image data, each sample, including the BSA-added sample, can be observed and analyzed as spots. The intensity of each spot area should be obtained, and subsequently, the data plotted on a graph with the amount of the standard protein on the horizontal axis and the fluorescent area intensity on the vertical axis. In the Examples, since the Y-axis intercept of the blank sample is background, the test sample data are extrapolated to the X-axis, which data point corresponds to the fluorescent area intensity at 0 ppm concentration of the BSA sample. The X-axis intercept of the test sample data indicates the protein concentration in the test sample. When BSA was used as the standard protein, at least 0.1 ppm of BSA was detected. For solution samples, the detection sensitivity can be directly applied as is. On the other hand, the detection sensitivity of solid samples depends on the solubility of the samples. The sample to be analyzed is dissolved in a solvent which provides the highest solubility, so that the highest detection sensitivity to the sample can be obtained.

In the method of the present invention, acids and alkalis may be removed after proteins have been adsorbed onto the membranes. Therefore, the method is also superior in that it is not limited as to the type of solvents.

Even the amino acids tyrosine and cystine, and the like, which are neutral and have a low solubility, can have an increased solubility when they are acidified.

Recently, in America (US) and also in Japan, foods and food additives that include genetically-modified organisms and plants have come onto the market, but deep-seated consumer anxiety still exists about GMOs (genetically-modified organisms). In the future, it will be increasingly necessary to quantify or detect the protein remaining in food additives produced from GMO, at sensitivity as high as possible, so as to guarantee the quality of products.

Furthermore, the field of regeneration medical treatment has become of interest recently, since the field uses the most advanced medical technology for regenerating human tissues and organs in vitro and transplanting them. Further development of this field in the future is increasingly expected. Since heterologous proteins are often allergenic, protein-free mediums are generally used for regenerating such tissues or organs. Accordingly, methods and means for analyzing or quantifying protein in such mediums at sensitivity as high as possible are desired for regeneration applications. Other applications include the determination of protein in medicaments, cosmetics, supplements (nutrient auxiliary agents, nutrient auxiliary foods), and healthy foods at high-sensitivity, and will be much more important in the future.

The present invention is described below more concretely with reference to the following non-limiting Examples.

### EXAMPLE 1

### Quantification of Protein in Food Additives, Such as Amino Acid Supplements

Devices: The following devices are used.

Micro-96-well simultaneous filtration apparatus (Atto's AE-6190 Model); Fluorescent image analyzer (Amersham Biotech's Fluor Imager 595 or Tyhoon 8600); PVDF membrane (Millipore's PVDF SEQUENCING MEMBRANE, Immobilon-P^{SQ}); Shaker (Tokyo Rika's MULTI-SHAKER MMS); Clean draft (manufactured by Yamato Kagaku);

Aspirator; Poly-messflask (100 ml); Siliconized Eppendorf-tube (1.5 ml); Siliconized chips (250, 1000 μl); and Micropipetter.

Reagents: The following reagents are used.

Fluorescent staining solution (Molecular Probe's Ruby protein blot stain, 200 ml bottle); BSA preparation (Sigma's PROTEIN STANDARD 1000 ppm); Hydrochloric acid (Kanto agaku's ultra-high-purity reagent, 250 ml bottle); Acetic acid (Junsei Kagaku's special grade reagent, 500 ml bottle); and Methanol (manufactured by Junsei Kagaku, for high-performance liquid chromatography, 3 liters bottle).

Experiment protocol: The experiment protocol, including the analysis protocol and experiment operation is below.

Analysis Protocol:

1. Washing of devices (washing of wells)

↓

2. Preparation of reagents (3.6% HCl, fixing solution)

↓

3. Preparation of samples

(Dissolution of amino acid supplement, dilution of BSA preparation, addition of the preparation to amino acid)

↓

4. Cutting and activation of PVDF membrane

↓

5. Dipping in 3.6% HCl for 30 minutes

↓

6. Setting membrane to blotter, and washing it

↓

7. Addition of sample to membrane

↓

8. Washing of membrane

↓

9. Drying of membrane

↓

10. Fixation of protein on membrane

↓

11. Washing of membrane

↓

12. Staining of membrane

↓

13. Washing of membrane

↓

14. Measurement and analysis with fluorescent imager

Experiment Operation:

Instruments of sample well, sealing gasket and gasket-holding plate are washed with pure water.

Preparation of Reagents:

2-1. 3.6% hydrochloric acid: 10 ml of 36% hydrochloric acid (Kanto Kagaku's ultra-high-purity reagent, 250 ml bottle) is taken with a micropipetter, and put into a 100-ml volumetric flask of polymeric material, and pure water is added thereto to make 100 ml.

2-2. Fixing solution: 10 ml of methanol (manufactured by Junsei Kagaku, for high-performance liquid chromatography, 3 liters bottle) and 7 ml of acetic acid (Junsei Kagaku's special grade reagent, 500 ml bottle) are taken with a micropipette, and put into a 100-ml poly-messflask, and pure water is added to 100 ml.

Preparation of Samples:

3-1. Weigh and dissolution of amino acid: The following amino acid supplements are each dissolved in 3.6% hydrochloric acid to a concentration of 120 mg/ml. (1.5-ml siliconized Eppendorf-tube is used.) L-Asp, (L-Cys)2, L-Glu, L-Tyr, L-Trp, L-Gln, L-Asp-Na, L-Glu-Na. L-Phe is dissolved in 3.6% hydrochloric acid to a concentration of 70 mg/ml.

3-2. Preparation of standard reagent (BSA): BSA is diluted with 3.6% hydrochloric acid to a concentration of 0, 0.1, 0.2, or 0.5 μg/ml. “0 μg/ml” is 3.6% hydrochloric acid.

3-3. Preparation of amino acid solution (SA) for test sample: Test samples are each diluted with an amino acid solution so that the BSA concentration in SA could be 0, 0.1, 0.2 or 0.5 μg/ml. “0 μg/ml” is amino acid solution.

Cutting and activation of PVDF membrane:

A PVDF membrane is cut with scissors into a piece of approximately 12 cm×9 cm, and its front and back faces are marked top right with a pencil so as to be differentiated from each other. Methanol is put into a vessel, and the membrane piece is dipped therein for 1 minute.

Dipping in 3.6% HCl for 30 minutes:

Hydrochloric acid is put into a vessel, and the activated PVDF membrane is dipped in 3.6% hydrochloric acid for 30 minutes.

Setting membrane to blotter, and washing it:

The membrane is set in a blotter, and 350 μl of 3.6% hydrochloric acid is applied thereto. Under suction with an aspirator, this is washed. An outline of the blotting apparatus (dot blotting apparatus) used herein is shown in FIG. 1.

Applying of sample to membrane:

350 μl of SA is applied to the membrane, and sucked with an aspirator.

Washing of membrane:

350 μl of 3.6% hydrochloric acid is applied to the membrane, and the membrane is washed with it under suction with an aspirator. Next, 350 μl of pure water is applied to the membrane thus washed, and the membrane is washed with it under suction with an aspirator.

Drying of membrane:

The membrane is released from the blotter (blotting apparatus), and dried using a clean draft chamber to avoid contamination for 1 hour.

Fixation of protein on membrane:

The fixing solution is put into a vessel for its exclusive use, and the dry membrane is floated on the fixing solution with its transfer surface facing down. Using a shaker, this is fixed for 15 minutes. (The speed of the shaker is 50 rpm.)

Washing of membrane:

Pure water is put into a vessel. The membrane is floated on the pure water with its transfer surface facing down. This is washed for 5 minutes, using a shaker. (The speed of the shaker is 50 rpm.) The membrane is taken out, and pure water in the vessel is exchanged, and the membrane is again washed. The washing operation with pure water is repeated 4 times.

Staining of membrane:

A staining solution is put into a vessel. The membrane is floated on the staining solution with its transfer surface facing down. This is stained for 15 minutes, using a shaker. (The speed of the shaker is 50 rpm.)

Washing of membrane:

After 15 minutes, the membrane is taken out, and pure water is put into the vessel. The membrane is floated on the pure water with its transfer surface facing down. This is washed for 1 minute, using a shaker. (The speed of the shaker is 50 rpm.) The membrane is taken out, and pure water in the vessel is exchanged, and the membrane is again washed. The washing operation with pure water is repeated 2 times.

Measurement and analysis with fluorescent imager:

14-1. Method of using fluorescent imager: Measurement conditions and protocol are described below.

Measurement Conditions:
Emission Filter: Rox61OBP30
PMT: 415
Laser: Green (532 nm)
Sensitivity: Normal
Measurement Protocol:
   (a) The cover of the body is opened, and the PVDF membrane is put on the glass plate. At this stage, the stained surface of the membrane is made to face down (on the glass side).
   (b) A non-fluorescent glass plate is put on the membrane to press it.
   (c) The body cover is closed.
   (d) The scanning range is defined, and the measurement is started.
   (e) After the scanning is finished, the data are stored.
   (f) In case of sensitivity insufficiency or fluorescent saturation, after changing the PMT voltage value, the measurement is again carried out.

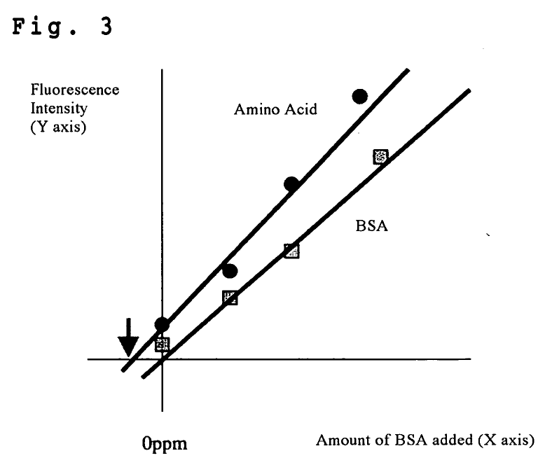

14-2. Imager: Using software purchased commercially (Amersham Biosciences UK), the image data are analyzed. As in FIG. 2, the spots are circled and the integral value of the fluorescent intensity in the circles is obtained.

Results:

An example of the image obtained according to the dot blotting method is shown in FIG. 2. In FIG. 2, the spot concentration of the BSA preparation increases with the increase in the amount thereof. A spot in added amount 0.1 μg/ml (0.1 ppm) of BSA is clearly observed. Therefore, this confirms that detection of at least 0.1 ppm protein is enabled. The same spot concentration change is also observed in the actual amino acid samples, and in addition, doughnut-like fluorescent circles concentric with sample spots are observed. This may be because the amino acid wetted around the wells while removed under reduced pressure would have reacted with the fluorescent reagent. Accordingly, in quantitative analysis, it has been decided that the product (volume) of the fluorescent intensity in the spot part is adopted. As a result, in the addition and recovery experiment of BSA and each amino acid, linearity is seen though the angle differs. The reasons why the angle differs depending on the type of amino acid are <1> there is a difference in the adsorbed amount of BSA to the instruments or the like, owing to the presence of the amino acid, and <2> the amino acid is adsorbed by BSA to remain. Accordingly, based on the BSA line and the intercept at the Y-axis of the blank (without BSA addition) data, which are measured on the same membrane, the intersections at the X-axis with of the data of each amino acid according to the standard addition method are defined as the protein concentration (see FIG. 3). The protein concentration in the actual samples is calculated according to the following formula, using the dilution ratio of each amino acid.

Protein concentration in actual sample
=(protein concentration in solution obtained according to standard addition method)×(dilution ratio).
Dilution ratio: 14.3 times for Asp, Cys, Glu, Phe, Tyr, Gln, Trp; and 8.3 times for other amino acids.

As a result, at least 0.1 ppm of the bovine serum albumin (BSA), and at least 1 ppm of the amino acid in terms of the concentration thereof in each sample can be detected.

FIG. 3 shows a method of calculating the protein concentration in each amino acid sample according to a standard addition method, in which the arrow indicates the protein concentration in an amino acid sample solution.

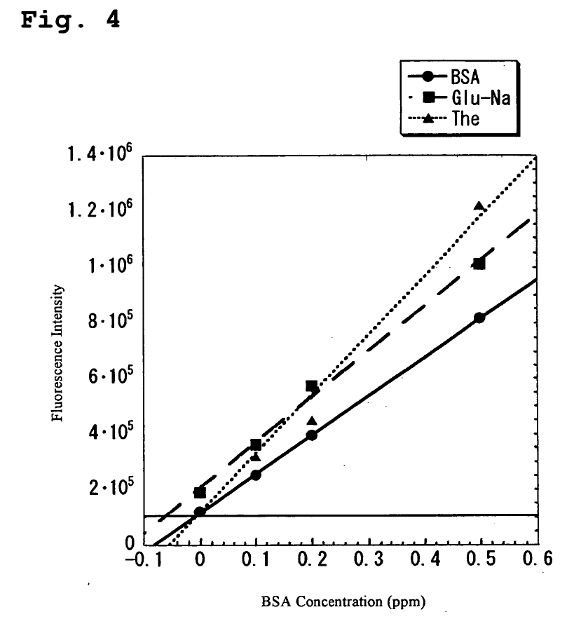

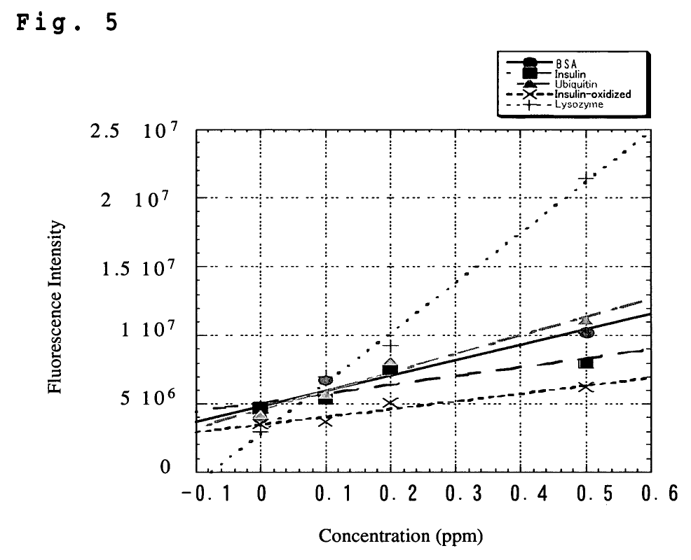

According to this method, 25 amino acids supplements, typically used as food additives, were analyzed. A part of the results are shown in FIG. 4; and a part of the quantification results of those 25 amino acids are shown in Table 1. In the 25 amino acid samples, the protein concentration was not more than 1 ppm (not more than 1 μg/g).

FIG. 4 shows measurement results (standard addition method) of protein in amino acid samples, GluNaH₂O (sodium L-glutamate monohydrate), and The (L-theanine), according to a dot blotting-fluorescence staining method.

TABLE 1
Quantification Results of protein in Amino Acids Samples

| | | Analysis Result |
|---|---|---|
| | | according to Dot |
| | | Blotting-Fluorescent |
| Abbreviations | Designation | Staining method |
| L-Ala | L-alanine | not more than 1 ppm |
| (L-Cys)₂ | L-cystine | not more than 1 ppm |
| L-GluNaH₂O | sodium L-glutamate | not more than 1 ppm |
| | monohydrate | |
| L-Ile | L-isoleucine | not more than 1 ppm |
| L-Leu | L-leucine | not more than 1 ppm |
| L-Thr | L-threonine | not more than 1 ppm |
| L-Tyr | L-tyrosine | not more than 1 ppm |
| L-Val | L-valine | not more than 1 ppm |
| L-CysHClH₂O | L-cysteine hydrochloride | not more than 1 ppm |
| | monohydrate | |
| L-Gln | L-glutamine | not more than 1 ppm |
| L-Trp | L-tryptophane | not more than 1 ppm |
| L-Arg | L-arginine | not more than 1 ppm |
| DL-Ala | DL-alanine | not more than 1 ppm |
| L-The | L-theanine | not more than 1 ppm |

The above-mentioned results confirm that, according to the method, trace amounts of protein in amino acid samples can be analyzed with high sensitivity. In particular, dot blotting is most effective when a PVDF membrane is used as a membrane for protein fixation thereon, with a fluorescent staining reagent. As a result of 25 amino acid samples using this method, it has been determined that the protein concentration in all these samples is not more than 1 ppm.

In this Example, the amino acid concentration is kept constant. However, in case of amino acids having higher solubility, by making the amino acid concentration high, the detection sensitivity can be further increased.

### EXAMPLE 2

### Molecular Weight Range of Detectable Protein

Proteins having a large molecular weight become allergens easily, and such high-molecular-weight proteins are readily detected in ELISA. Alternatively, even proteins having a lower molecular weight may become allergens, so it is desirable that proteins and peptide having a lower molecular weight be able to be detected with high sensitivity. According to the method of the present invention, proteins having a varying molecular weights, i.e., BSA (66 kDa), lysozyme (14 kDa), ubiquitin (8.6 kDa), insulin (5.7 kDa) and oxidized insulin B chain (3.5 kDa), were tested for the minimum limit of detection and the quantification ability thereof. In place of BSA in Example 1, each protein was suitably diluted and its calibration curve was formed according to the dot blotting-fluorescence staining method. As a result (see FIG. 5), all proteins had calibration curves of high linearity, and their minimum limit of detection was 0.1 ppm (but the minimum limit of detection of oxidized insulin B chain was only 0.2 ppm). This result confirms a detection of low molecular weight proteins with high-sensitivity.

As described hereinabove, the present invention provides a method of analyzing protein content. According to the method, trace amounts of proteins, such as allergens, in food, drinks, food additives, medicaments, cosmetics, feed, etc. can be simply analyzed with high sensitivity. Accordingly, the present invention has wide applications in various industrial fields, especially in foods, medicaments, cosmetics, feeds, etc..

While the invention has been described in detail with reference to preferred embodiments thereof, it will be apparent to one skilled in the art that various changes can be made, and equivalents employed, without departing from the scope of the invention. Each of the aforementioned documents, as well as the parent application, PCT/JP03/09305, and the foreign priority document, JP2002-217099, are incorporated by reference herein in its entirety.

1. A method of analyzing a sample for allergenic substances comprising
a) dissolving said sample in an aqueous solution,
b) fixing protein in said solution onto a membrane, and
c) detecting the level of said protein by fluorescence staining,
wherein the level of said protein indicates the presence of said allergenic substance.

2. The method of claim 1, wherein the presence of said allergenic substance is determined by detection of trace amounts of said protein.

3. The method of claim 2, wherein 0.1 ppm of said protein is able to be detected.

4. The method of claim 1, wherein said membrane is selected from the group consisting of nitrocellulose and PVDF.

5. The method of claim 1, wherein said sample is selected from the group consisting of food, drinks, food additives, medicaments, feed.

6. The method of claim 5, wherein said sample is an amino acid.

7. The method of claim 6, wherein the solubility of said amino acid is improved by dissolving said amino acid in an aqueous solution with an acidic pH.
